(19) United States
(12) Patent Application Publication
(10) Pub. No.: US 2009/0137968 A1
(43) Pub. Date: May 28, 2009

(54) Apparatus for Controlled Blood Regurgitation Through Tricuspid Valve

(75) Inventor: Dan Rottenberg, Haifa (IL)

Correspondence Address: THE NATH LAW GROUP, 112 South West Street, Alexandria, VA, 22314, US

(73) Assignee: TRANSPID LTD., Caesarea (IL)

(21) Appl. No.: 12/226,429

(22) PCT Filed: Apr. 18, 2007
(86) PCT No.: PCT/IL2007/000489

§ 371 Date: Oct. 17, 2008

Related U.S. Application Data
(60) Provisional application No. 60/792,905, filed on Apr. 19, 2006.

(51) Int. Cl. A61M 25/00; A61M 31/00

(52) U.S. Cl. 604/264; 604/508; 604/509

(57) ABSTRACT

A device for reducing the pulmonary pressure by forcing controllable tricuspid valve regurgitation, using a catheter assembly (10), comprising, a catheter tube (12) having a proximal end and a distal end, the distal end fitted with a navigation balloon (18) inflatable through an inflation lumen. The catheter assembly further comprises an expander (20) surrounding a section of the catheter and a manipulating sleeve (30) slidingly enveloping a portion of the tube extending rearwards from the expander and forming a gap between the tube and the sleeve, whereby the expander is adapted to radially expand and contract through manipulation of the sleeve. The tube further comprises a first pressure lumen (26), a second pressure lumen (28), and a third pressure conduit (32); wherein the first and second pressure lumens and the third pressure conduit terminate at pressure outlet couplings of the tube's proximal end.

### FIELD OF THE INVENTION

This invention relates to a catheter assembly and a method for inducement and monitoring of a tricuspid regurgitation for the treatment of different heart failure conditions such as lung edema.

### BACKGROUND OF THE INVENTION

Congestive Heart Failure (CHF) is a condition characterized by the inability of the heart to pump blood at a rate needed to maintain adequate blood flow throughout the body. CHF most commonly occurs as a result of ischemic heart disease, myocardial infarction, cardiomyopathy, or valvular disorders. In most cases, the left ventricle (which pumps oxygenated blood to the body) fails, while the right ventricle (which pumps deoxygenated blood to the lungs) continues to function normally. When this occurs, the pulmonary arterial pressures increase and fluid begins to exude from the pulmonary capillaries into the pulmonary interstitium and eventually into the alveoli, the tiny air sacs where gas exchange takes place. Fluid in the alveoli impedes gas exchange across the alveolar membranes, which then lowers the blood oxygenation. The resulting hypoxemia has a detrimental effect on the cardiac muscle, which requires large amounts of oxygenated blood to function normally, and results in an even more profound heart failure. This cycle, if left untreated, continues until the patient succumbs, in effect, drowning in his own pulmonary fluids.

During the course of CHF induced lung edema, some patients develop a condition named tricuspid regurgitation, whereby a faulty valve allows blood to reflux backward through the valve into the right atrium. It has been found that those patients who develop tricuspid regurgitation during the course of CHF actually experience improvements in their pulmonary symptoms. Such edema improvement can theoretically be achieved without any significant reduction in cardiac output, if regurgitation level and reduction of pulmonary pressures are controlled, and not reduced below certain levels that effect left ventricle cardiac output (known in the art as Frank-Sterling curve).

Several types of catheters and methods for inserting catheters through the tricuspid valve are disclosed in the prior art.

U.S. Pat. No. 5,207,228 discloses a catheter for monitoring heart function, which comprises a catheter tube having a plurality of lumens. The catheter includes an inflatable balloon at a distal tip of the catheter tube for positioning the catheter in a wedged position within a pulmonary artery within the heart of a patient. Dual injectate ports are formed in a side wall of the catheter tube. Each port communicates with a respective injectate lumen carried in the catheter tube. When inserted into the heart of a patient, either the first or second injectate port is positioned within the desired distance from the tricuspid valve for thermodilution depending upon the size of the heart. Thermodilution to obtain cardiac output and/or right heart ejection fraction is implemented by injecting injectate through the lumen associated with the port properly positioned relative the tricuspid valve and discharged through the corresponding port.

In U.S. Pat. No. 5,509,428 a method and apparatus are disclosed for the controlled inducement of tricuspid regurgitation for the purpose of treating congestive heart failure. The apparatus is comprised of a catheter onto which is affixed an expandable device. The catheter is percutaneously inserted into the patient's body, and then moved within the central venous system until the end of the catheter traverses the tricuspid valve of the heart. The expandable device on the distal end of the catheter is then radially expanded to hold open the leaflets of the tricuspid valve. The catheter advancement and positioning as disclosed in this patent is performed through fluoroscopic observation.

The EP0363117 discloses a balloon-tipped, flow-directed catheter with a position-monitoring port is adapted to be positioned in the outflow tract of the right ventricle proximal to the pulmonic valve of the heart, which in most adult patients would be approximately 8 cm from the distal tip of the catheter. By measuring the pressure at the position-monitoring port, the position of the catheter tip and the balloon in the pulmonary artery can be monitored, and checked before inflating the balloon to measure pulmonary wedge pressure at the distal port. A right ventricular pressure reading indicates that the balloon is in a proper position in the pulmonary artery for measuring wedge pressure. A pulmonary artery pressure reading indicates that the position-monitoring port has crossed the pulmonic valve and that the balloon has migrated distally to a position that may not be safe for inflating the balloon to measure wedge pressure. In such a case, the catheter should be gradually withdrawn until a right ventricular pressure is obtained.

U.S. Pat. No. 5,034,001 discloses a vascular catheter having an expandable cage mounted on the distal end of a tubular member which is radially expanded and contracted by means of a control wire which is secured to the distal end of the expandable cage. The control wire extends through a first inner lumen within the tubular member which extends along essentially the entire length thereof. A second inner lumen is provided in the distal portion of the tubular member which has a proximal port at least 15 but not more than 60 cm from the distal end of the catheter and a distal port which opens into the interior of the expandable cage. A guidewire or a low-profile steerable catheter is slidably disposed within the second lumen and a tubular member such as a slightly expanded coil through the expandable cage interior to facilitate the rapid exchange of the catheter. The catheter assembly is particularly adapted to hold open a blood vessel after a vascular procedure therein such as an angioplasty.

A catheter which includes means to measure local pressure at two or more points along the catheter body is described in WO0113789. The points are preferably located in two different pressure areas, more preferably across a valve in a vessel, organ or similar.

### SUMMARY OF THE INVENTION

The present invention is concerned with a device and method for precise positioning of an expander-catheter system used for inducing controllable tricuspid regurgitation. Using the catheter in accordance with the present invention facilitates in-situ position confirmation without the need for radioscopic or other observation and thus simplifies cardiac procedures.

Even more so the method and device according to the present invention enable real-time monitoring and confirmation of correct positioning of the device and the actual cardiac output. A procedure according to the present invention may be used whilst the patient is seated, which is a requirement in certain cardiac positions.

According to a first aspect of the present invention there is provided a catheter assembly comprising a catheter tube having a proximal end and a distal end, configured to pass axially through a venous system, said distal end fitted with a navigation balloon inflatable through an inflation lumen partially extending through a major portion of the tube; an expander surrounding a section of said catheter, extending between a rear end and a fore end, and having a blood flow path, the assembly further comprising a manipulating sleeve having a front end and a rear end slidingly enveloping a portion of the tube extending rearwards from the expander and forming a gap between the tube and said sleeve, whereby said expander is adapted to radially expand and contract through manipulation of the sleeve; the tube further comprising a first pressure lumen extending from the distal end for measuring a first pressure point, a second pressure lumen extending from adjacent in front of the fore end of the expander for measuring a second pressure point and a third pressure conduit extending from adjacent the fore end of the expander for measuring a third pressure point, said first and second pressure lumens and the third pressure conduit terminate at the tubes proximal end at pressure outlet couplings.

According to one embodiment of the present invention the expander normally extends flush over the tube and its fore end is secured over the tube and its rear end is slidably displaceable thereon; and where a front end of the manipulating sleeve is engaged with a rear end of the expander to thereby axially displace said rear end of the expander so as to facilitate its expansion and contraction.

According to another embodiment the expander is normally biased to spontaneously expand over the tube, where its fore end and its rear end are affixed over the tube and whereby the manipulating sleeve extends over the expander and retains it at its retracted configuration whereby upon retraction of the sleeve the expander is gradually exposed to thereby expand its exposed portion.

According to another aspect of the present invention, a method for aiding in positioning a cardiac catheter assembly is provided, the method comprising the steps of providing a catheter assembly, comprising:

a tube having a proximal end and a distal end, configured to pass axially through a venous system and heart ventricle:

said tube's distal end fitted with a navigation balloon inflatable through an inflation lumen partially extending through a major portion of the tube;

an expander surrounding a section of said tube, extending between a rear end and a fore end, and having a blood flow path;

a manipulating sleeve having a front end and a rear end slidingly enveloping a portion of the tube extending rearwards from the expander and forming a gap between the tube and said sleeve, whereby said expander is adapted to radially expand and contract through manipulation of the sleeve;

the tube further comprising a first pressure lumen extending from the distal end for measuring a first pressure point;

a second pressure lumen extending from adjacent in front of the fore end of the expander for measuring a second pressure point; and

a third pressure conduit extending from adjacent the rear end of the expander for measuring a third pressure point;

said first and second pressure lumens and the third pressure conduit terminate at the tubes proximal end at pressure outlet couplings.

For obtaining tricuspid regurgitation the method further comprises the following steps:

deflating the navigation balloon when in the pulmonary artery;

radially expanding the expander of the catheter, through manipulation of the manipulating sleeve, so that the expander holds open the leaflets of the tricuspid valve;

inducing tricuspid regurgitation by holding open of said leaflets of the tricuspid valve;

monitoring the patient's pulmonary pressure, the ventricle pressure and the atrial pressure and cardiac output, adjusting the degree of tricuspid regurgitation to affect reduction of pulmonary pressures and improvement in the lung edema; and

removing the catheter from the patient's body.

Any one or more of the following embodiments may be featured in the device according to the present invention or the method using same:
the sleeve is pushed forwards to expand the expander.
the front end of the sleeve is articulated to the rear end of the expander.
the expander is a wire mesh.
the expander has an effective area sufficient to expand an artery valve.
the length of the effective area is of a length sufficient to expand a tricuspid valve to facilitate blood flows from the right ventricle to the right atrium.
the length of the effective area is between about 5 mm to 140 mm.
the third pressure conduit is formed within a gap between the tube and the sleeve.
a sealing ring is fitted over the sleeve adjacent the rear end of the expander to prevent fluid passage through the expander into the gap.
an inlet port of the third pressure conduit is formed adjacent the front end of the sleeve and behind the sealing ring, to facilitate fluid pressure pick-up through the third pressure conduit. Said inlet port is a displaceable port axially shiftable together with the sleeve's distal end and the expander's proximal end.
the first pressure point is obtained in a pulmonary artery, the second pressure point is obtained in a right ventricle and the third pressure point is obtained in a right atrium.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIG. 1A is a general view of a cardiac catheter according to an embodiment of the present invention

FIG. 1B is a schematic longitudinal section of the catheter assembly according to one embodiment of the present invention;

FIG. 1C is a cross section along line C-C in FIG. 1A;

FIG. 1D is a cross section along line D-D in FIG. 1A;

FIG. 1E is a cross section along line E-E in FIG. 1A.

FIG. 2 is a schematic longitudinal section of a catheter assembly according to another embodiment of the present invention;

FIGS. 3A and 3B illustrate the expander portion of the catheter assembly according to FIG. 2 in a collapsed and the expanded configurations respectively;

FIGS. 4A to 4C illustrate consecutive steps of expanding the expander of the catheter assembly between a collapsed position (FIG. 4A) and fully expanded position (FIG. 4C) according to additional embodiment of the present invention;

FIG. 5 is a schematic representation of a heart fitted with the catheter assembly of the present invention; with the expander at its collapsed configuration;

FIG. 6 is similar to FIG. 5 wherein the catheter assembly is in its expanded configuration; and

FIGS. 7A to 7C are a waveform representation of the pressure measurements as received from ports P_{I}, P_{II} and P_{III}, respectively.

FIG. 8 is a schematic illustration of an expander according to an embodiment of the present invention.

### DETAILED DESCRIPTION OF EMBODIMENTS

The present invention discloses an apparatus and a method for controlled inducement of tricuspid regurgitation for the purpose of treating congestive heart failure.

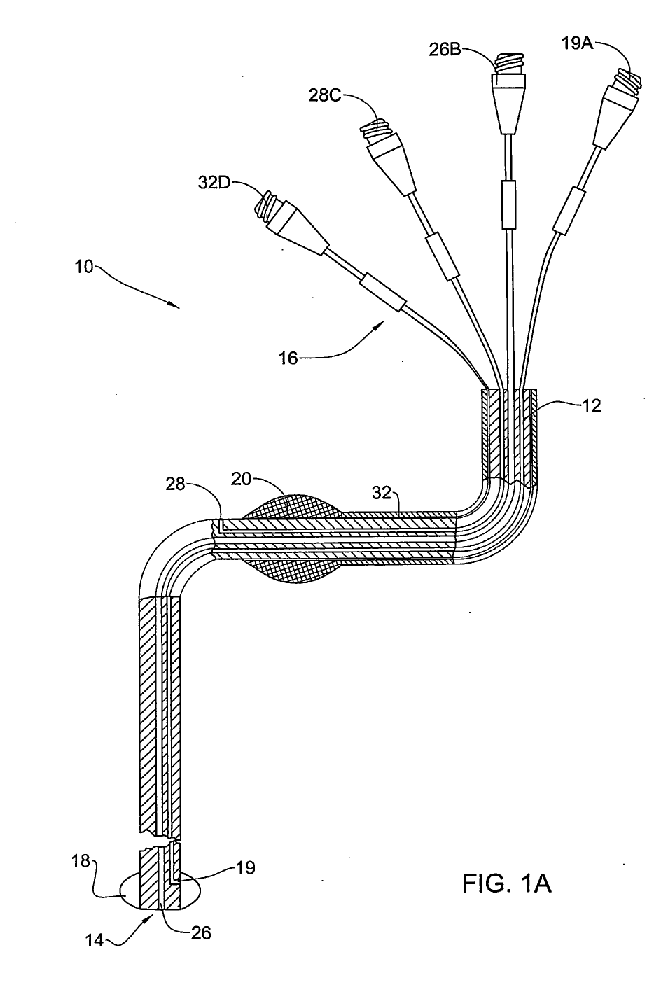

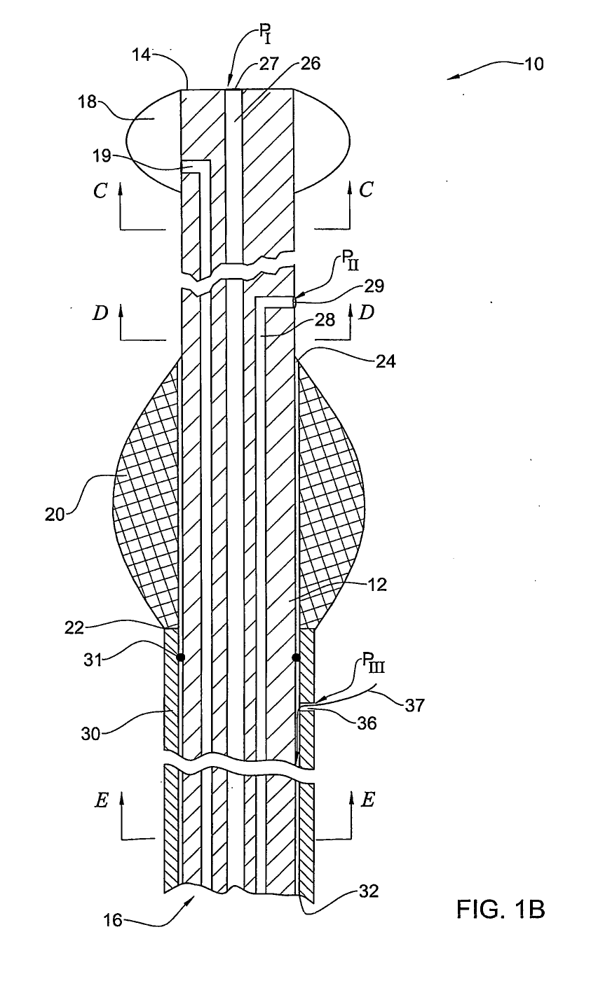

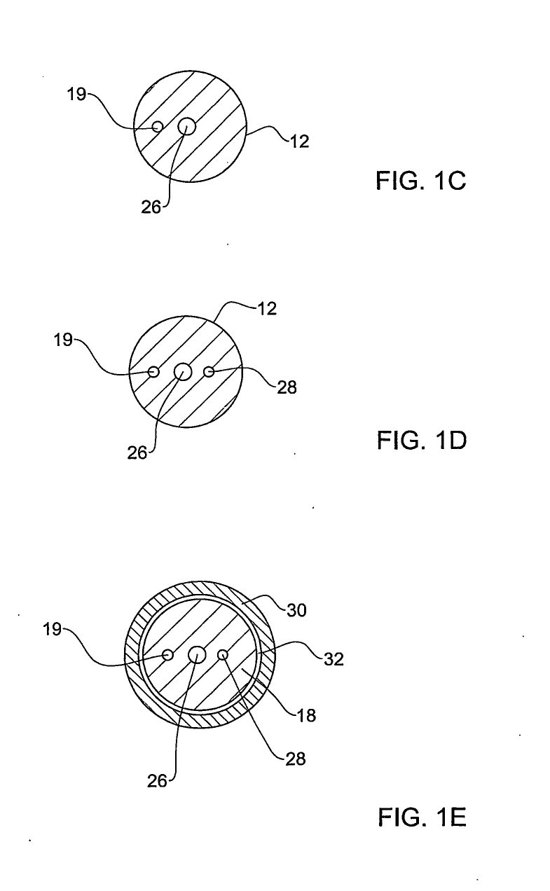

Attention is first directed to FIGS. 1A and 1B of the drawings, illustrating a catheter assembly generally designated 10, in accordance with one embodiment of the present invention, comprising, a tube 12 having a proximal end 16 and a distal end 14, said catheter assembly configured to pass axially through the venous system and right ventricle. The distal end 14 of the catheter tube 12 is fitted with a navigation balloon 18 inflatable through an inflation lumen 19 extending through a major portion of the catheter tube 12.

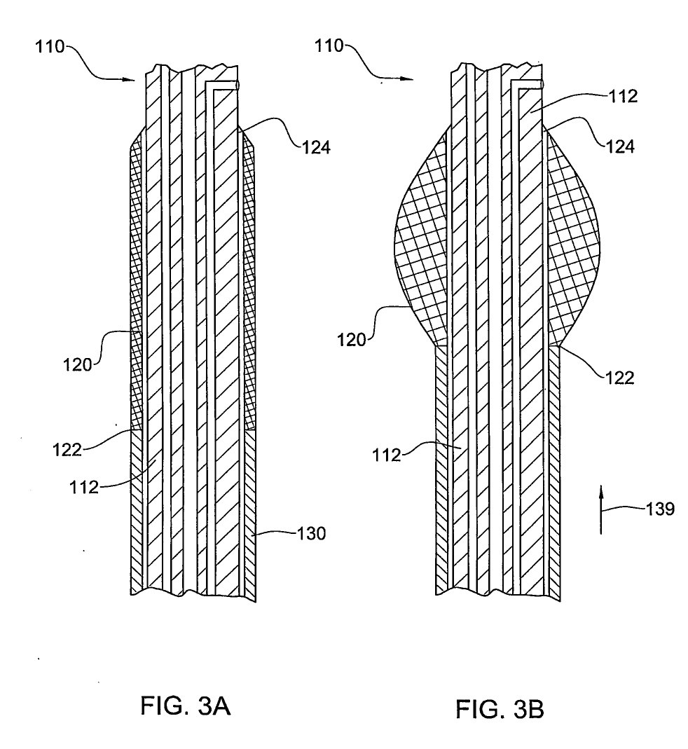

The catheter assembly 10 of the present invention further comprises an expander 20 surrounding a section of said catheter tube 12 and extending between a rear end 22 and a fore end 24, the expander 20 adapted to allow blood flow therethrough. According to the present embodiment, at its contracted configuration (see FIG. 3A), the expander 20 extends substantially flash over the tube 12 where its fore end 24 is secured over the tube 12 and its rear end 22 is slidably displaceable over the tube. The rear end 22 of the expander 20 is engaged with a manipulating sleeve 30 slidingly enveloping a portion of the tube 12 extending rearwards from said rear end 22 whereby the sleeve 30 is adapted to displace said rear end 22 of the expander 20 so as to facilitate expansion and contraction of the expander 20 (FIGS. 3A and 3B, respectively). The sleeve 30 envelopes the tube such that a gap 32 is formed between the tube 12 and said sleeve 30, said gap facilitating a third pressure conduit 32 as will be discussed hereinafter.

The catheter tube 12 further comprises a first pressure lumen 26 extending from or adjacent the distal end 14 towards the proximal end 16, adapted to measure a first pressure point referred to as P_{I} through a first pressure port 27. A second pressure lumen 28 extends from adjacent in front of the fore end 24 of the expander 20 towards the proximal end 16, adapted to measure a second pressure point referred to as P_{II} through a second pressure port 29.

According to this embodiment, the manipulating sleeve 30 is fitted with a sealing ring 31 proximal the rear end 22 of the expander 20, thus preventing blood flow through the expander 20 into the gap 32.

The third pressure conduit 32 extends between the sealing ring 31 towards the proximal end 16 with an inlet port 36 for measuring a third pressure point P_{III}. The inlet port 36 moves with the sleeve 30 when pulled or pushed relative to tube 12 (the inlet port displaces axially along the tube).

Attention is also directed to FIGS. 1C through 1E illustrating respective sections taken along the catheter 10 and schematically illustrating the configuration of the catheter.

Each of the pressure lumens 26, 28, and the third pressure conduit 32 extend towards pressure outlet coupling fitted at the proximal 16 of the catheter 10 fitted for connection to a pressure monitor, as will be discussed hereinafter. Likewise, the inflation lumen 19 extends towards an inflating port at the proximal end of the catheter assembly 16 for coupling to a pressure source, e.g. syringe (not shown). The lumens 19, 26, 28 and the pressure conduit 32 communicate with proximal end connector tubes 19A, 26B, 28C and 32D, respectively, as shown in FIG. 1A. Tube 19A leads to connector (not shown) for connection to an inflation apparatus, such as an inflation syringe. Bacteria-filtered carbon dioxide is one type of inflation medium beneficial due to its rapid absorption in the blood in the event of balloon rupture within the circulation.

Tubes 26B, 28C and 32D connect to a pressure measurement apparatus (not shown) in order to monitor pressures at the pressure ports 27, 29 and 36.

For sake of clarity, further embodiments of the present invention will be discussed with reference to figures, wherein like components are designated with like numerals shifted by hundred.

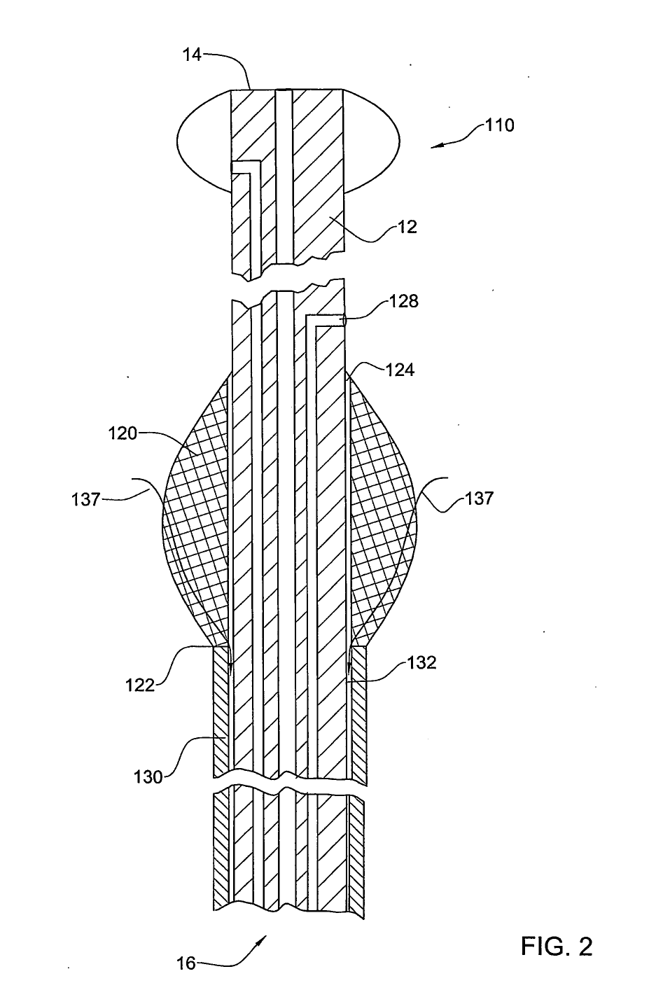

FIG. 2 is another embodiment of a catheter assembly generally designated 110, substantially similar to the embodiment of FIGS. 1A-1E, however, with a fore end 124 of the expander 120 affixed to a tube 112 and whereby a manipulating sleeve 130 is engaged with the rear end 122 of the expander 120, such that a blood flow path (illustrated by arrow 137) extends through the expander 120 and a third pressure conduit 132 for measurement of the third pressure point P_{III}.

In FIGS. 3A and 3B a partial illustration of the catheter assembly 110 of FIG. 2 is shown. As can be seen in FIG. 3A the expander 120 is in its contracted configuration fitted substantially flash over the tube 112 with its fore end 124 secured to the tube 112 and its rear end 122 engaged with the manipulating sleeve 130, whereby in FIG. 3B the manipulating sleeve 130 has been pushed forwards in direction of arrow 139 with corresponding axial displacement of the rear end 122 (whilst its fore end 124 is fixed over the tube 112), such that the expander 120 radially expands. It is appreciated that the extent of radial expansion depends on an amount of the axial displacement of the rear end 122.

The expander 20, 120 according to the present invention can be a wire mesh or any other suitable configuration fitted for controlled radial expansion whilst allowing fluid flow therethrough at its expanded configuration. The expander may be made of metal wire, fabric, plastic material etc. the expander, according to any of its embodiments may be made of or coated by a bio-compatible material.

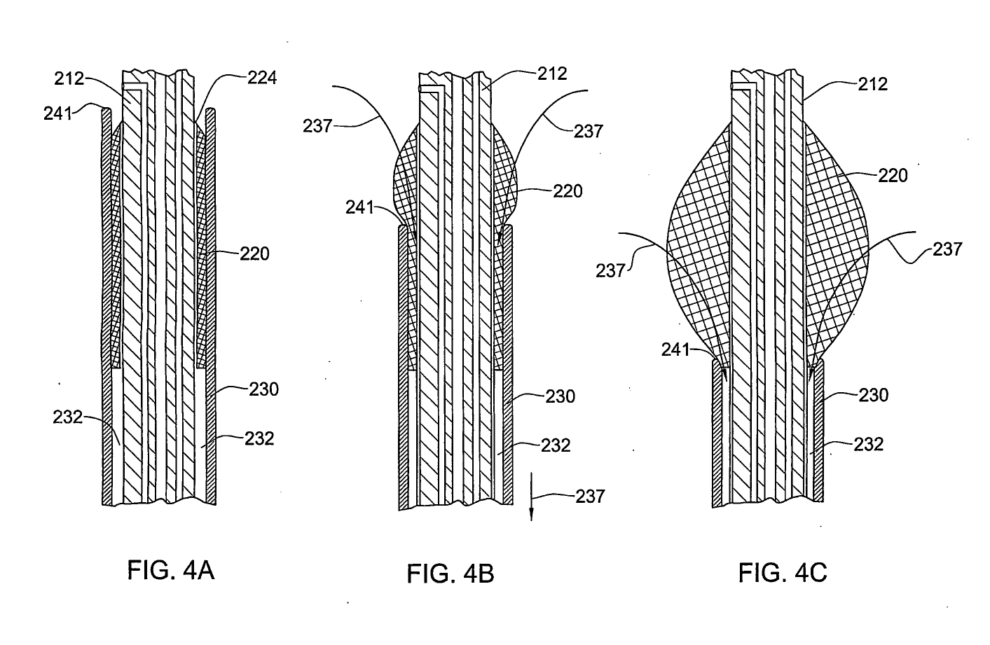

In FIGS. 4A to 4C an additional embodiment of the present invention is illustrated, this embodiment is substantially similar to the previous embodiments, wherein the main differences reside in the expander and the expansion mechanism as will be discusses hereinafter. The configuration of the pressure lumens is substantially similar to the previous embodiment as already discussed.

As can be seen in FIG. 4A, both the fore end 224 and the rear end 222 of an expander 220 are secured over a tube 212. The expander 220 is made of memory shape alloy and is normally biased into spontaneous expansion. However, a manipulating sleeve 230 embraces the expander 220 such that it prevents spontaneous expansion thereof. The manipulating sleeve 230 is slidingly displaceable over the tube 212. Upon axial withdrawal of the sleeve 230 in a direction of the arrow 237 (FIG. 4B), the expander 220, begins to spontaneously expand until a maximum expansion is achieved upon complete retraction of manipulating sleeve 230 (FIG. 4C)

According to this embodiment a third pressure conduit 232 extends from the fore end 241 of the manipulating sleeve 230, though the expander 220 to the proximal end 216 (not seen) of the catheter assembly 210 such that the blood enters the gap 232 between the manipulating sleeve 230 and the tube 212.

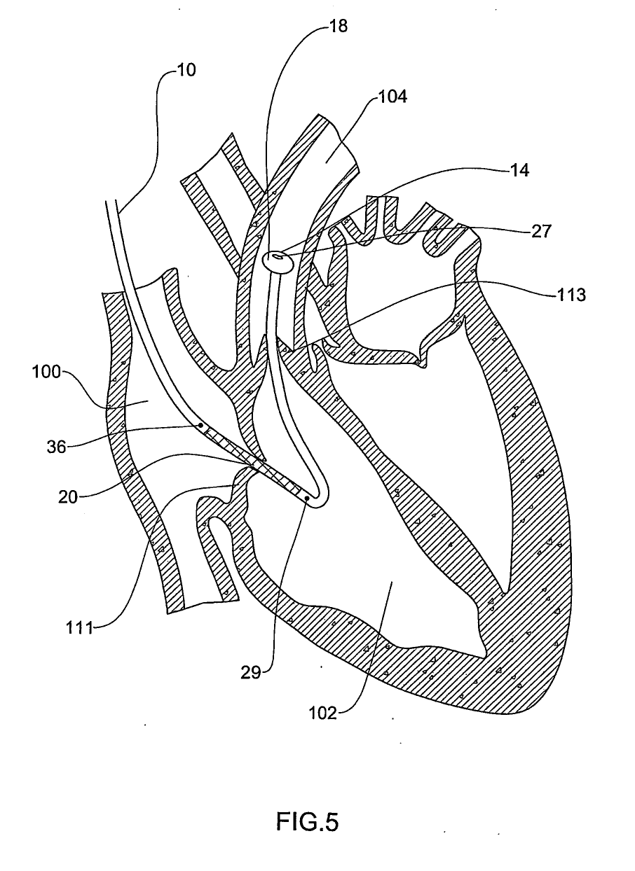

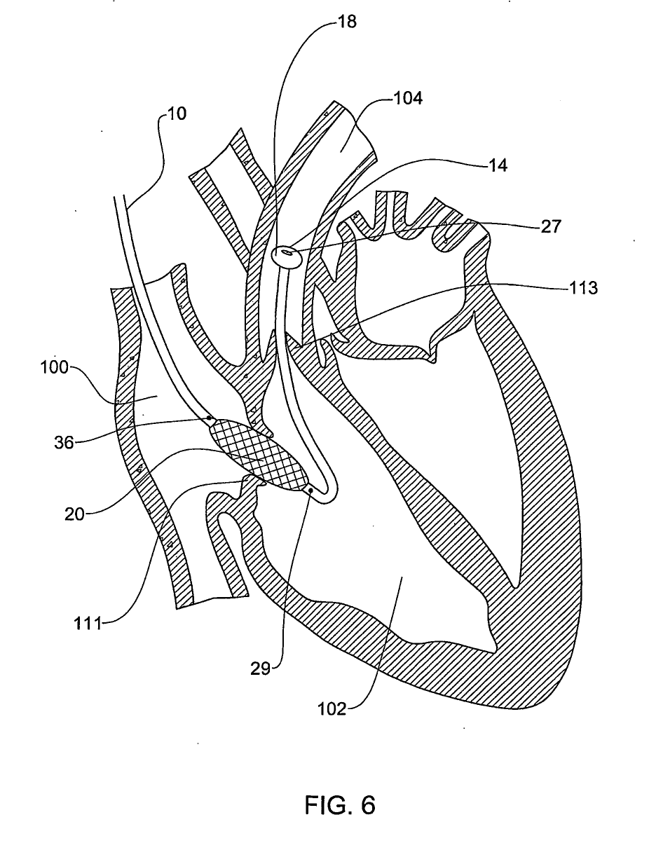

FIGS. 5 and 6 illustrate a particular method for utilizing the catheter assembly according to the present invention, for controlled inducement of the tricuspid regurgitation. The method is exemplified with reference to FIGS. 1A-3B, though it is applicable using a catheter assembly according to any of other embodiments as well.

The catheter assembly 10 is initially inserted into the patient's venous system with the expander 20 at its contracted configuration and with the balloon 18 deflated (not shown). The assembly 10 can be introduced through a large vein, often the internal jugular, subclavian, or femoral veins.

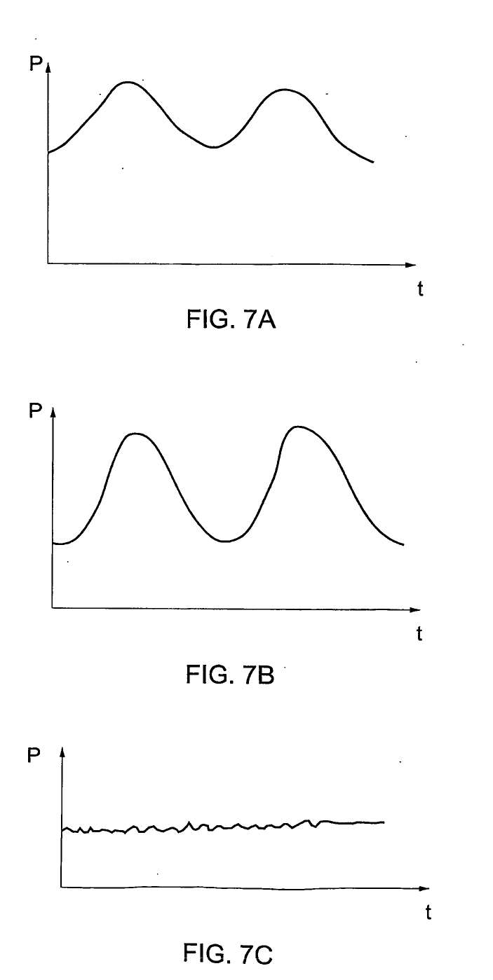

The catheter assembly 10 is advanced through the right atrium 100. Once within the right atrium 100 the flow-directed navigation balloon 18 is inflated through the inflation lumen 19 (e.g. by aid of predetermined volume syringe). Hydraulics of the blood flow advance the inflated balloon 18 through the tricuspid valve 111 into the right ventricle 102 and thus carries and navigates the distal end 14 of the catheter assembly 10 through the pulmonary valve 113 and into the main pulmonary artery 104. The position of the balloon 18 within the pulmonary artery 104 is confirmed through monitoring pressure point P_{I} through pressure lumen 26 (yielding a pressure graph as in FIG. 7A).

It is now required to position the expander 20 within the vicinity of the tricuspid valve 111 for efficient and controlled expansion thereof.

Additional pressure measurements are now taken through the second pressure lumen 28 and third pressure conduit 32 to confirm positioning of the expander 20 at its desired position within the tricuspid valve 111. The position of the expander 20 is confirmed through measurement of pressure points P_{II} and P_{III}, wherein P_{II} indicates the pressure in the right ventricle (FIG. 7B) and P_{III} indicates the pressure at the right atrium (FIG. 7C). The pressure ports 29 and 36 are respectively positioned adjacent before and behind the expander 20, at all times (i.e. at its collapsed and partially or fully expanded positions), thus providing definite pressure indicia corresponding with the pressure upstream and downstream the valve 111, confirming that the expander 20 is positioned within the tricuspid valve 111.

In case pressure measurements obtained at the second pressure measurement point P_{II} and the third measurement point P_{III} do not fall within the expected values of the pressure measurements of the right ventricle 102 and right atrium 100 (FIGS. 7B and 7C, respectively), the catheter assembly 10 may easily be slightly advanced or retracted in order to obtain correct positioning confirmed by the respective pressure measurements.

Once the desired position for the expander 20 has been obtained, the balloon 18 may be deflated.

After correct position of the expander 20 within the valve 111 has been confirmed, the expander 20 is slowly expanded to a desired rate to facilitate blood reflux backward through the valve 111 to the right atrium 100, condition also known as tricuspid regurgitation. The intention of causing the regurgitation is to lower the pulmonary pressures elevated as a result of inability of the failed left heart to respond and compensate for acute changes in the cardiovascular system, such as after-load elevation.

This procedure can advantageously be performed whilst a torso of the patient is substantially upright (e.g. seated) this being a significant advantage with patients with sever lung edema, and further without the need in observations such as radiology or fluoroscopic observation etc. which often require undesired mobilization of the patient, availability of radiology equipment and specially trained staff.

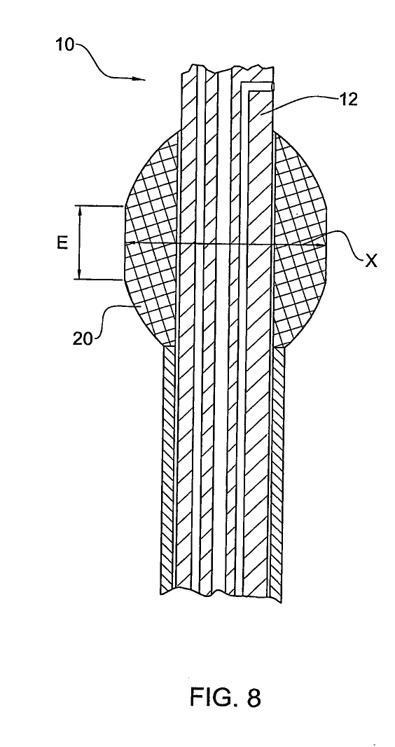

According to a particular embodiment of the present invention the length of the catheter assembly 10 is approximately 140 cm. The distance between the inflatable balloon 18 and the second pressure port 29 is in the range of 15-30 cm (according to a specific embodiment, it is 20 cm). The expander 20 according to the present invention in its contracted configuration is in the range of approximately 1 to 12 cm in length and wherein the length of an effective area for keeping the leaflets of the tricuspid valve in their open configuration is in the range of approximately 1 to 7 cm. The term “effective area” refers to an area having a mean diameter in the range of about 3-20 mm (see FIG. 8).

As can be seen in FIG. 6 after verifying pressure measurements to confirm correct positioning of the expander 20, the expander is expanded. As described above this could be performed through various manipulating arrangements as discussed in connection with the embodiments hereinabove.

The rate of expansion of the expander 20 is monitored through measurements performed by pressure measuring ports 27, 29 and 36, whereby the tricuspid valve 111 leaflets are held open and blood reflux is allowed from the right ventricle 102 into the right atrium 100 thus causing a tricuspid regurgitation. During this process patient's pulmonary arterial pressure and right ventricle pressure are closely monitored as well as the patients cardiac output. The degree of tricuspid regurgitation is thus controlled in order to obtain required pulmonary pressures

Whilst some embodiments have been described and illustrated with reference to some drawings, the artisan will appreciate that many variations are possible which do not depart from the general scope of the invention, mutatis, mutandis.

1. A catheter assembly, comprising:
a catheter tube having a proximal end and a distal end, configured to pass axially through a venous system and the right ventricle;
said distal end fitted with a navigation balloon inflatable through an inflation lumen partially extending through a major portion of the tube;
an expander surrounding a section of said catheter, extending between a rear end and a fore end, and having a blood flow path;
a manipulating sleeve having a front end and a rear end slidingly enveloping a portion of the tube extending rearwards from the expander and forming a gap between the tube and said sleeve, whereby said expander is adapted to radially expand and contract through manipulation of the sleeve;
the tube further comprising a first pressure lumen extending from the distal end for measuring a first pressure point through a first pressure port;
a second pressure lumen extending from adjacent in front of the fore end of the expander for measuring a second pressure point through a second pressure port; and
a third pressure conduit extending from adjacent the rear end of the expander for measuring a third pressure point through an inlet port;
said first and second pressure lumens and the third pressure conduit terminate at the tubes proximal end at pressure outlet couplings.

2. A catheter assembly according to claim 1, wherein the expander normally extends flush over the tube and where its fore end is secured over the tube and its rear end is slidably displaceable thereon; and where a front end of the manipulating sleeve is engaged with a rear end of the expander to thereby axially displace said rear end of the expander so as to facilitate its expansion and contraction.

3. A catheter assembly according to claim 1, wherein the expander is normally biased to spontaneously expand over the tube, where its fore end and its rear end are affixed over the tube; and whereby the manipulating sleeve extends over the expander and retains it at its retracted configuration whereby upon retraction of the sleeve the expander is gradually exposed to thereby expand its exposed portion.

4. A catheter assembly according to claim 2, whereby the sleeve is pushed forwards to expand the expander.

5. A catheter assembly according to claim 2, wherein the front end of the sleeve is articulated to the rear end of the expander.

6. A catheter assembly according to claim 1, wherein the expander is a wire mesh.

7. A catheter assembly according to claim 1, wherein the expander has an effective area sufficient to expand a heart valve.

8. A catheter assembly according to claim 7, wherein the length of the effective area is of a length sufficient to expand a tricuspid valve leaflets to facilitate blood flows from the right ventricle to the right atrium.

9. A catheter assembly according to claim 8, wherein the length of the effective area is between about 1 cm to 12 cm.

10. A catheter assembly according to claim 1, wherein the third pressure conduit is formed within a gap between the tube and the sleeve, and the inlet port displaces with the sleeve.

11. A catheter assembly according to claim 2, wherein a sealing ring is fitted over the sleeve adjacent the rear end of the expander to prevent fluid passage through the expander into the gap.

12. A catheter assembly according to claim 11, wherein an inlet port of the third pressure conduit is formed adjacent the front end of the sleeve and behind the sealing ring, to facilitate fluid pressure pick-up through the third pressure conduit.

13. A catheter assembly according to claim 1, wherein the first pressure point is obtained in a pulmonary artery, the second pressure point is obtained in a right ventricle and the third pressure point is obtained in a right atrium.

14. A method for aiding in positioning a cardiac catheter assembly, the method comprising:
a. providing a catheter assembly, comprising:
a tube having a proximal end and a distal end, configured to pass axially through a venous and right ventricle system, said distal end fitted with a navigation balloon inflatable through an inflation lumen partially extending through a major portion of the tube;
an expander surrounding a section of said catheter extending between a rear end and a fore end, and adapted to allow blood to reflux past the expander;
a manipulating sleeve slidingly enveloping a portion of the tube extending rearwards from the expander, whereby said expander is adapted to expand and contract through manipulation of the sleeve;
the tube further comprising a first pressure lumen extending from the distal end for measuring a first pressure point through a first pressure port;
a second pressure lumen extending adjacent in front the fore end of the expander for measuring a second pressure point through a second pressure port; and
a third pressure conduit extending from adjacent the rear end of the expander for measuring a third pressure point through an inlet port;
said first and second pressure lumens and the third pressure conduit terminate at the tubes proximal end at a pressure outlet couplings;
b. inserting the catheter assembly into a patient's venous system;
c. advancing the catheter assembly through the patient's venous system into a patient's heart;
d. inflating the navigation balloon to be directed by blood flow from the right atrium into the pulmonary artery area and positioning the expander in proximity of a tricuspid valve; and
e. conforming location of the expander within the tricuspid valve by confirming pulmonary pressure through the first pressure point, conforming right ventricular pressure through the second pressure point and confirming the right atrial pressure through the third pressure point.

15. A method according to claim 14, further comprising the following steps:
a. deflating the navigation balloon when in the pulmonary artery;
b. radially expanding the expander of the catheter, through manipulation of the manipulating sleeve, so that the expander holds open the leaflets of the tricuspid valve;
c. inducing tricuspid regurgitation by holding open of said leaflets of the tricuspid valve;
d. monitoring the patient's pulmonary pressure, the right ventricle pressure and the atrial pressure and cardiac output, adjusting the degree of tricuspid regurgitation to affect the reduction of pulmonary pressures and improvement in the lung edema; and
e. removing the catheter from the patient's body.

16. A method of claim 14, wherein the step of measuring the patient's pulmonary pressure, the ventricle pressure, the atrial pressure and cardiac output is periodically performed during the medical procedure.

17. A method according to claim 14, wherein the expander is normally contracted over the tube and where its fore end is secured over the tube and its rear end is slidably displaceable thereon; and where a front end of the manipulating sleeve is engaged with a rear end of the expander so as to facilitate its expansion and contraction.

18. A method according to claim 14, wherein the expander is normally biased to spontaneously expand over the tube, where its fore end and its rear end are affixed over the tube; and whereby the manipulating sleeve extends over the expander and retains it at its retracted configuration whereby upon retraction of the sleeve the expander is gradually exposed to thereby expand its exposed portion.

19. A method according to claim 17, whereby the sleeve is pushed backwards to expand the expander.

20. A method according to claim 14, wherein the front end of the sleeve is articulated to the rear end of the expander.

21.-27. (canceled)
